(19) United States
(12) Patent Application Publication
(10) Pub. No.: US 2025/0281028 A1
(43) Pub. Date: Sep. 11, 2025

(54) ENDOSCOPE AND METHOD FOR CONTROLLING THE ENDOSCOPE

(71) Applicant: MAX-PLANCK-GESELLSCHAFT ZUR FÖRDERUNG DER WISSENSCHAFTEN E.V., München (DE)

(72) Inventors: Metin Sitti, Tübingen (DE); Martin Phelan, Tübingen (DE)

(73) Assignee: MAX-PLANCK-GESELLSCHAFT ZUR FÖRDERUNG DER WISSENSCHAFTEN E.V., München (DE)

(21) Appl. No.: 19/099,843

(22) PCT Filed: Aug. 1, 2023
(86) PCT No.: PCT/EP2023/071339

§ 371 Date: Jan. 30, 2025

(30) Foreign Application Priority Data
Aug. 2, 2022 (DE) 202022104403.1
Aug. 2, 2022 (DE) 202022104405.8
Aug. 2, 2022 (LU) LU502623
Aug. 2, 2022 (US) 17816774
Aug. 2, 2022 (US) 17816779
Dec. 9, 2022 (EP) 22212545.2
Dec. 9, 2022 (LU) LU503167

(51) Int. Cl. A61B 1/00; A61B 18/00; A61B 18/08

(52) U.S. Cl. CPC A61B 1/00158; A61B 1/00006; A61B 1/0005; A61B 1/00133; A61B 18/085; A61B 2018/00041; A61B 2018/00446; A61B 2018/00577; A61B 2018/00595; A61B 2018/00982

(57) ABSTRACT

An endoscope and a method for controlling a movement of the endoscope in a magnetic field, the endoscope comprising a tip, a set of coils surrounding the tip, and power wires arranged to supply the set of coils with electrical energy, wherein the set of coils comprises four side coils arranged around the tip such that a straight line extending orthogonally to a longitudinal direction of the tip crosses a center of the respective side coil.

### CROSS REFERENCE TO RELATED APPLICATIONS

This application is a national stage of international patent application PCT/EP2023/071339, filed on Aug. 1, 2023 and designating the U.S., which claims priority to US patent application U.S. Ser. No. 17/816,774, filed on Aug. 2, 2022, and to German patent application DE 20 2022 104 403.1, filed on Aug. 2, 2022, and to US patent application U.S. Ser. No. 17/816,779, filed on Aug. 2, 2022, and to German patent application DE 20 2022 104 405.8, filed on Aug. 2, 2022, and to Luxembourgish patent application LU502623, filed on Aug. 2, 2022, and to European patent application EP 22 212 545.2, filed on Dec. 9, 2022, and to Luxembourgish patent application LU503167, filed on Dec. 9, 2022, all of which are hereby incorporated by reference in their entireties.

### TECHNICAL FIELD

The present disclosure relates to an endoscope and a method for controlling a movement of the endoscope in a magnetic field.

Additionally or alternatively, the present disclosure may relate to a data processing device configured to carry out the method at least partly.

Additionally or alternatively, a computer program may be provided, wherein the computer program comprises instructions which, when the program is executed by a computer, e.g., the data processing device, cause the computer to carry out the method at least partly.

Additionally or alternatively, a (non-tangible) computer-readable (storage) medium may be provided, wherein the computer-readable medium comprises instructions which, when executed by a computer, cause the computer to carry out the method at least partly.

### BACKGROUND

Any discussion of the related art throughout the specification should in no way be considered as an admission that such related art is widely known or forms part of common general knowledge in the field.

US 2007/088197 A1 describes a method of navigating a medical device having a changeable magnetic moment within an operating region within a patient. The method includes applying a navigating magnetic field to the operating region with an external source magnet, and changing the direction of the magnetic moment in the medical device to change the orientation of the medical device in a selected direction within the operating region. The magnetic moment of the medical device can be created by one or more electromagnetic coils, in which case the magnetic moment can be changed by changing the current to the coil. Alternatively, the magnetic moment of the medical device can be created by one or more permanent magnets, in which case the magnetic moment can be changed by mechanically or magnetically manipulating the permanent magnet.

U.S. Pat. No. 6,304,769 B1 describes an apparatus comprising a directable device adapted for insertion into a patient. The device comprises a substrate wrapped with a multi-coil package comprising overlapping or overlaid coaxial coils, which package is adapted for conducting multiple, independent electric currents such that when the currents are passed through the multi-coil package a local directable magnetic moment is created. The apparatus comprises a means for selectively applying electric currents through the multi-coil package to orient the substrate and a means external to the patient for generating a magnetic field to which the directable device is exposed, wherein the local directable magnetic moment is at a controllable angle with respect to the substrate to orient the substrate in the magnetic field.

U.S. Pat. No. 6,594,517 B1 describes a method and apparatus for generating a controlled torque of a desired direction and magnitude in an object within a body, such as a catheter through a blood vessel in a living body, by producing an external magnetic field of known magnitude and direction within the body, applying to the object a coil assembly including preferably three coils of known orientation with respect to each other, and controlling the electrical current through the coils to cause the coil assembly to generate a resultant magnetic dipole interacting with the external magnetic field to produce a torque of the desired direction and magnitude.

Widespread use of noninvasive medical imaging modalities in surgeries such as magnetic resonance imaging (MRI), X-ray, and ultrasound (US) has enabled the deployment of micron-sized surgical tools.

Neuroendoscopy is a minimally invasive technique to visualize and treat areas of the central nervous system including the skull, brain, and spine. Currently, neuroendoscopic systems are used to treat many different kinds of cases, including intraventricular lesions, craniosynostosis, spinal lesions, and skull base tumors. To this end, both rigid and flexible endoscopes are utilized in surgical operations.

However, the standard rigid endoscopic tools have drawbacks such as limited field of view and risk of blunt force trauma. Neuronavigation may improve existing techniques, especially among patients with intraventricular pathologies. Additionally, flexible endoscopes have a better mobility range which may be critical in complex anatomies like ventricles.

Although the introduction of neuroendoscopy into surgical operations has made significant improvements over other traditionally invasive surgical approaches, there is still room for improvement including improved precision and less trauma to surrounding tissue.

The main challenge in active endoscope design is transmitting force and torque through the soft slender body to actuate the endoscope tip. Some systems use tendon-based force transmission. However, researchers have proposed many different alternative actuation mechanisms, such as multi-backbone, concentric tubes, pneumatics, smart materials, hydraulics, magnetics, or hybrid approaches to overcome certain drawbacks of tendon-based systems.

Integration to existing medical imaging modalities is another crucial part of device design. X-ray imaging is currently the gold standard for real-time visualization during minimally invasive surgeries. Radiopaque endoscopes can be visualized easily in vascular structures filled with contrast agents. However, it is hard to visualize the effect of the intervention on soft tissue with X-ray imaging due to the poor soft-tissue contrast. Therefore, there is a growing interest in MRI-guided minimally invasive interventions due to the high soft-tissue contrast of MRI images for visualizing blood vessels (such as within the brain of a human being) and tissue response as well as no ionizing radiation, real-time tool tracking capabilities, and physiological measurement capabilities (e.g., MRI thermometry, diffusion, and perfusion).

However, MRI scanners impose new constraints on medical device design and actuation. First, the permanent high magnetic field limits material choices to nonmagnetic materials for device construction to avoid unintended magnetic force and torque. Second, large nonmagnetic metal objects cause imaging susceptibility artifacts. Third, the MRI scanner's radio-frequency (RF) pulses can induce heating within conductive materials found in medical tools.

Therefore, there are numerous studies on developing MR-compatible actuation techniques for device steering.

MRI-driven actuation offers signific advantages over the aforementioned techniques due to its scalability, safety, response time (nearly instantaneous), accuracy (other methods experience nonlinearities in actuation), and degrees of freedom (DoF). In addition, MRI-driven actuation can utilize imaging gradient coils user-controlled to generate spatial field gradients for steering a wireless robot or magnetic endoscope tip in 3D.

However, embedding magnetic elements to endoscopes for gradient steering introduces significant MR image distortion and additional endoscope weight and bulkiness.

Another MRI-driven actuation approach is mounting microcoils on the endoscope tip for Lorentz force-based endoscope steering using manually-wound or laser-machined microcoils.

Moreover, the image distortion can be controlled since the image artifacts only occur when coils are activated.

Magnetically-assisted cateterization using microcoils has been shown to be faster than manual navigation using MR imaging guidance for larger angles and comparable to X-ray guidance.

However, microcoil-based Joule heating effects have been a major design concern. Prior research has shown tissue thermal injury occurs above local temperatures of 44° C. Studies have indicated using currents inputs above 300 mA (1.2 W) can lead to vessel thrombus, vacuolization, and medial hemorrhage. Potential solutions have included integrating heat dissipation mechanisms, such as alumina to the endoscope tip and passing saline coolant through the microcoil tip, or regulating current to less than 300 mA (1.2 W) with less than 1 min activation times.

However, such solutions introduce additional weight and bulkiness to the endoscope tip, require flow through the endoscope, limit working channel size, and constrain the time necessary for active steering in the workspace.

It may be an object of the present invention to overcome or ameliorate at least one of the disadvantages of the prior art, and/or to provide a useful alternative.

### SUMMARY

Provided is an endoscope, optionally a neuroendoscope. The endoscope comprise a tip, optionally comprising a working channel extending through the tip of the endoscope. The endoscope comprises a set of coils surrounding the tip. The endoscope comprises power wires arranged to supply the set of coils with electrical energy. The set of coils comprises four side coils arranged around the tip such that a straight line standing orthogonal on a longitudinal direction of the tip crosses a center of the respective side coil, i.e., the four side coils are arranged around the tip such that a magnetic flux direction of the four side coils points towards a center line of the tip.

### BRIEF DESCRIPTION OF THE DRAWINGS

FIG. 1 shows schematically, in a perspective view an endoscope.

FIG. 2 shows schematically side coils of the endoscope of FIG. 1.

FIG. 3 shows schematically a cross sectional view of a tip of the endoscope of FIG. 1.

FIG. 4 shows schematically, in a top view a grasper of the endoscope of FIG. 1 in a closed state.

FIG. 5 shows schematically, in a top view the grasper of the endoscope of FIG. 1 in an open state.

FIG. 6 shows a flow diagram of a method for controlling a movement of the endoscope of FIG. 1 in a magnetic field.

FIG. 7 shows a flow diagram of a further method for controlling a movement of the endoscope of FIG. 1 in a magnetic field.

### DESCRIPTION OF EXEMPLARY EMBODIMENTS

As required, detailed embodiments are disclosed herein; however, it is to be understood that the disclosed embodiments are merely exemplary of the invention that may be embodied in various forms. The figures are not necessarily to scale, and some features may be exaggerated to show details of particular components. Therefore, specific structural and functional details disclosed herein are not to be interpreted as limiting, but merely as a basis for the claims and as a representative basis for teaching one skilled in the art to variously employ the present invention.

A first and a second one of the side coils may be connected in series. Additionally or alternatively, a third and a fourth one of the side coils may be connected in series. Additionally or alternatively, the first and the second one of the side coils may be arranged on opposite sides of the tip. Additionally or alternatively, the third and the fourth one of the side coils may be arranged on opposite sides of the tip and in-between the first and the second one of the side coils.

A turn number of at least one of the four side coils may be between 2 and 40 (i.e, including 2 and 40). Optionally, the turn number of at least one of the four side coils may be between 4 and 30 (i.e, including 4 and 30). Optionally, the turn number of at least one of the four side coils may be 7 (seven).

The set of coils may comprise at least one axial coil arranged around the tip such that a straight line extending in parallel to the longitudinal direction of the tip crosses a center of the at least one axial coil.

At least one of the coils of the set of coils may be manufactured using laser machining, laser lithography and/or manually wound.

At least one of the coils of the set of coils may have, an optionally rectangular, Archimedean spiral coil design.

At least one of the coils of the set of coils may have an in-plane design.

The four side coils may be arranged on the same, optionally flexible, circuit board.

The endoscope may comprise an end effector connected to the tip of the endoscope. The end effector may comprise a further set of coils for actuating the end effector.

The end effector may be configured to be actuated by applying a current to the further set of coils. At least one of the methods described below may comprise actuating the end effector by applying a current to the further set of coils.

The end effector may comprise a grasper with two jaws connected pivotably to each other. Additionally or alternatively, the further set of coils may comprise at least one side coil arranged at each one of the two jaws, respectively.

Actuating the end effector may comprise opening and/or closing the jaws of the grasper by applying the current to the further set of coils. The opening may be a movement where the jaws of the grasper are moved away from each other by turning them around the pivotable connection and the closing may be a movement where the jaws of the grasper are moved towards each other by turning them around the pivotably connection.

The end effector may be configured such that ablating and/or killing tissue, optionally comprising tumor cells, using the Joule heating caused by the current supplied to the further set of coils for actuating the end effector, optionally for opening and closing the grasper, is possible. At least one of the methods described below may comprise ablating and/or killing tissue, optionally comprising tumor cells, using the Joule heating caused by the current supplied to the further set of coils for actuating the end effector, optionally for opening and closing the grasper.

The tip of the endoscope may comprise a camera. Additionally or alternatively, the tip of the endoscope may comprise an illumination device, optionally comprising a light emitting diode (LED). Additionally or alternatively, the tip of the endoscope may comprise an opening of an irrigation channel extending through the endoscope.

Additionally or alternatively the disclosure is directed to the use of a grasper of an endoscope, optionally a neuroendoscope, for cauterization. The endoscope comprises a tip, an end effector connected to the tip, and a set of coils arranged at the end effector. The cauterization comprises actuating the end effector by applying a current to the set of coils, and ablating and/or killing tissue, optionally comprising tumor cells, using the Joule heating caused by the current supplied to the set of coils for actuating the end effector. That above given description with respect to the endoscope applies mutatis mutandis to the use for cauterization thereof and vice versa.

A first method for controlling a movement of the above described endoscope is provided, wherein the method comprises determining a torque that needs to be applied onto the endoscope such that the endoscope carries out the movement.

The first method comprises determining a minimum current that needs to be supplied to each coil of the set of coils, respectively, to reach the determined torque by solving an optimization problem.

The first method comprises supplying the determined minimum current to each coil of the set of coils, respectively, such that the endoscope carries out the movement.

The optimization problem (for determining the minimum current) may be defined as follows:

$${I}^{*} = {arg min}_{I} {‖ {τ}_{coils} - {τ}_{des} ‖}^{2} + α {‖ I ‖}_{R}^{2}$$

wherein:
I may represent a current supplied to each coil of the set of coils, respectively,
τ_coils may represent a total torque generated by the set of coils when being supplied with the current I,
τ_des may represent the torque that needs to be applied onto the endoscope such that the endoscope carries out the movement, and
R may represent a resistance of each coil of the set of coils.

Determining the torque may comprise solving a further optimization problem to minimize the torque to be applied onto the endoscope such that the endoscope carries out the movement.

The endoscope may comprise a flexible substantially rod-shaped portion. Additionally or alternatively, the movement may comprise a deformation of said rod-shaped portion resulting in a movement of a tip of said rod-shaped portion.

Solving the further optimization problem may comprise determining the deformation of said rod-shaped portion that is needed for the movement of the tip of said rod-shaped portion from an actual location to a desired location such that a torque that is required for the deformation of said rod-shaped portion is minimized. Additionally or alternatively, solving the further optimization problem may comprise determining the torque that needs to be applied onto the endoscope such that the endoscope carries out the movement to be equal to the torque that is required for the deformation of said rod-shaped portion.

The deformation that is needed for the movement of the tip of said rod-shaped portion from the actual location to the desired location may be determined using a model, optionally a Cosserat model, depicting the nonlinear dynamics of said rod-shaped portion.

Additionally or alternatively, a second method for controlling a movement of the above described endoscope in a magnetic field may be provided. The above given description with respect to the first method applies mutatis mutandis to the second method, and vice versa.

The second method comprises determining a torque that needs to be applied onto the endoscope such that the endoscope carries out the movement. Determining the torque that needs to be applied onto the endoscope comprises solving an optimization problem to minimize the torque that needs to be applied onto the endoscope such that the endoscope carries out the movement.

The second method comprises determining a current that needs to be supplied to the set of coils to reach the determined torque.

The second method comprises supplying the determined current to the set of coils such that the endoscope carries out the movement.

The endoscope may comprise a flexible substantially rod-shaped portion. Additionally or alternatively, the movement may comprise a deformation of said rod-shaped portion resulting in a movement of a tip of said rod-shaped portion.

Solving the optimization problem (for minimizing the torque) may comprise determining the deformation of said rod-shaped portion that is needed for the movement of the tip of said rod-shaped portion from an actual location to a desired location such that a torque that is required for the deformation of said rod-shaped portion is minimized. Additionally or alternatively, solving the optimization problem (for minimizing the torque) may comprise determining the torque that needs to be applied onto the endoscope such that the endoscope carries out the movement to be equal to the torque that is required for the deformation of said rod-shaped portion.

The deformation that is needed for the movement of the tip of said rod-shaped portion from the actual location to the desired location may be determined using a model, optionally a Cosserat model, depicting the nonlinear dynamics of said rod-shaped portion.

At least one of the above described methods may comprise receiving user input with respect to the movement via a user interface, optionally comprising a joy stick. The user interface may be connected directly or indirectly, e.g., via a control unit, to the endoscope.

At least one of the above described methods may comprise determining an actual position of the endoscope, optionally the tip thereof, using medical imaging. Additionally or alternatively, at least one of the above described methods may comprise an automated controlling of the movement based on the determined actual position.

At least one of the above described methods may comprise displaying an actual position of the endoscope, optionally a tip thereof, and/or a position of the endoscope, optionally a tip thereof, after carrying out the movement on a display device. The display device may be connected to a medical imaging device.

At least one of the above described methods may comprise displaying the actual position of the endoscope, optionally the tip thereof, and/or the position of the endoscope, optionally the tip thereof, after carrying out the movement with respect to a tissue, optionally of a human being or an animal, on the display device.

In at least one of the above described methods the magnetic field may be produced by a medical imaging device, optionally a magnetic resonance imaging device.

In at least one of the above described methods the magnetic field may be a static magnetic field.

Additionally or alternatively, a data processing device may be provided which is configured to carry out at least one of the above described methods at least partly.

Additionally or alternatively, a computer program may be provided, wherein the computer program may comprise instructions which, when the program is executed by a computer, e.g., the data processing device, cause the computer to carry out at least one of the above described methods at least partly.

Additionally or alternatively, a computer-readable (storage) medium may be provided, wherein the computer-readable medium comprises instructions which, when executed by a computer, cause the computer to carry out at least one of the above described methods at least partly.

In the following definitions of terms used in this description are given, wherein the respective description is just one possible specific definition out of many possible definitions of the respective term and is thus not intended to limit the scope of the disclosure to this specific definition.

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Where ever the phrase “for example”, “such as”, “including” and the like are used herein, the phrase “and without limitation” is understood to follow unless explicitly stated otherwise. Similarly “an example”, “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The term “about” when used in connection with a numerical value refers to the actual given value, and to the approximation to such given value that would reasonably be inferred by one of ordinary skill in the art, including approximations due to the experimental and or measurement conditions for such given value.

The terms “comprising” and “including” and “having” and “involving” (and similarly “comprises”, “includes”, “has”, and “involves”) and the like are used interchangeably and have the same meaning. Specifically, each of the terms is defined consistent with the common United States patent law definition of “comprising” and is therefore interpreted to be an open term meaning “at least the following”, and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The term “method” as used herein may include a computer-implemented method. The expression “computer-implemented method” covers claims which involve computers, computer networks or other programmable apparatus, whereby at least one feature is realised by means of a program. A computer-implemented method may be a method which is at least partly carried out by a data processing unit, e.g. a computer.

The term “controlling” may be defined as a process in a system in which one or more variables as input variables influence other variables as output variables due to the laws peculiar to the system. Additionally or alternatively, the term “controlling” may be defined as a process in which a variable, the controlled variable (the variable to be controlled), is continuously recorded, compared with another variable, the reference variable, and influenced in the sense of an adjustment to the reference variable.

The term “movement” may include any displacement or change of a position of a device, here the medical device, optionally the tip thereof. In one possible interpretation the movement may be a motion. A motion may be the phenomenon in which an object, here the medical device, optionally the tip thereof, changes its position with respect to space and time, optionally the magnetic field.

The term “determining” may include carrying out one or more mathematical operations in order to determine based on a given input in a given manner a desired output.

The term “torque” may be the rotational equivalent of a linear force. The term “torque” may be also referred to as the moment, moment of force, rotational force or turning effect. The torque may represent the capability of a force to produce change in the rotational motion of a body, such as the rode shaped portion of the medical device. The torque may be defined as the product of the magnitude of the force and the perpendicular distance of the line of action of the force from the axis of rotation. In three dimensions, the torque may be a pseudovector; for point particles, it is given by the cross product of the position vector (distance vector) and the force vector. The magnitude of torque of a rigid body may depend on three quantities: the force applied, the lever arm vector connecting the point about which the torque is being measured to the point of force application, and the angle between the force and lever arm vectors. The toque may be defined by the force that is applied to the tip of the medical device.

Instead of the term “current”, the term “electric current” may be used. An electric current is a stream of charged particles, such as electrons or ions, moving through an electrical conductor or space. It is measured as the net rate of flow of electric charge through a surface or into a control volume. The moving particles are called charge carriers, which may be one of several types of particles, depending on the conductor. In electric circuits the charge carriers are often electrons moving through a wire. In semiconductors they can be electrons or holes. In an electrolyte the charge carriers are ions, while in plasma, an ionized gas, they are ions and electrons. The SI unit of electric current is the ampere, or amp, which is the flow of electric charge across a surface at the rate of one coulomb per second. Electric currents create magnetic fields, which may be are used to actuate or move the medical device in the (external) magnetic field. In ordinary conductors, they cause Joule heating.

Instead of the term “coil”, the term electromagnetic coil may be used. An electromagnetic coil may be an electrical conductor such as a wire in the shape of a coil, spiral or helix. An electric current may be passed through the wire of the coil to generate a magnetic field. A current through any conductor creates a circular magnetic field around the conductor due to Ampere's law. One advantage of using the coil shape may be that it increases the strength of the magnetic field produced by a given current. The magnetic fields generated by the separate turns of wire all pass through the center of the coil and add (superpose) to produce a strong field there. The more turns of wire, the stronger the field produced may be and the stronger the effect of Joule heating may be.

An optimization problem may be described as the problem of finding substantially the best solution from all feasible solutions.

The endoscope may be a medical device. A medical device may be any device intended to be used for medical purposes. According to one possible definition a medical device may be an instrument, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in man or other animals, and/or intended to effect the structure or any function of the body of man or other animals, and which does not achieve its primary intended purposes through chemical action within or on the body of man or other animals and which is not dependent upon being metabolized for the achievement of its primary intended purposes. The term “medical device” may or may not include software functions. According to another possible definition the term “medical device” may mean any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings or animals for the purpose of diagnosis, prevention, monitoring, treatment or alleviation of disease, diagnosis, monitoring, treatment, alleviation of or compensation for an injury or handicap, investigation, replacement or modification of the anatomy or of a physiological process, and/or control of conception, and which does not achieve its principal intended action in or on the human and/or animal body by pharmacological, immunological or metabolic means, but which may be assisted in its function by such means.

Instead of the term “resistance”, the term “electrical resistance” may be used. The resistance multiplied by the current may be equal to the (electrical) voltage.

A rod-shaped portion may be a portion or part of the medical device that may have a substantially circular cross section. The rod-shaped portion may have cylindrical form wherein a height or length of the rod-shaped portion exceeds the diameter of the rod-shaped portion. The tip of the rod-shaped portion may be outermost part of the rod-shaped portion in a forward direction of the medical device. The tip of the rod-shaped portion may comprise a region around the circumference at or near the end of the outermost part of the rod-shaped portion. The rod-shaped portion may comprise or may be realized using a tube. The tip may be called an insertion tip of the medical device.

The Cosserat model may be based on the Cosserat rod theory. This approach may allow for a substantially exact solution to the static of a continuum robot, as it is not subject to any assumption. It solves a set of equilibrium equations between position, orientation, internal force and torque of the robot, here the medical device, optionally the rod-shaped portion thereof. Creating an accurate model that can predict the shape of a continuum robot allows to properly control the robot's shape.

An endoscope is a medical device that may be used as an inspection instrument. An endoscope may comprise an image sensor, an optical lens, a light source and/or a mechanical device, which is used to look deep into the body by way of openings such as the mouth or anus.

A neuroendoscope may be an endoscope configured to be used for neuroendoscopy which is a minimally invasive surgical technique that allows inspection (and optionally illumination) of angles in hidden parts of the surgical field, enabling optionally clear visualization and manipulation of anatomical structures. There are three main subdisciplines of endoscopic neurosurgery: intraventricular neuroendoscopy for the treatment of occlusive hydrocephalus and other lesions within and around the ventricular system; transnasal neuroendoscopy including the different endoscopic endonasal approaches for pituitary and further skull base pathologies; as well as transcranial endoscope-assisted microneurosurgery for various kinds of intracranial tumors, cysts and neurovascular lesions. However, it has to be noted that the present disclosure is not limited to these three fields.

A medical imaging device may be a device configured to be used for medical imaging. Medical imaging is the technique and process of imaging the interior of a body. Medical imaging may incorporate radiology, which uses the imaging technologies of X-ray radiography, magnetic resonance imaging, ultrasound, endoscopy, elastography, tactile imaging, thermography, medical photography, and/or nuclear medicine functional imaging techniques such as positron emission tomography (PET) and single-photon emission computed tomography (SPECT).

A magnetic resonance imaging device is a medical imaging device configured to be used in magnetic resonance imaging (MRI) which is a medical imaging technique that may be used in radiology to form pictures of the anatomy and the physiological processes of the body. MRI scanners use strong magnetic fields, magnetic field gradients, and radio waves to generate images of the organs in the body.

A working channel may be a channel, optionally having a circular form, arranged in the rod-shaped portion of the medial device. The working channel may extend throughout the whole rod shaped portion. The working channel may be used to transport a (medical) device, a gas and/or a fluid from one end of the medical device, optionally located outside of the body, to the tip of the rod-shaped portion. The working channel may house or accommodate devices permanently and/or temporarily, such as a camera, an electrical wire and/or a light source, at least partly.

Cauterization may be defined as the action of burning body tissue using heat, to stop an injury from bleeding or getting infected, and/or to remove harmful cells.

In the following a solution to the Lorentz force-induced heating concern without using active cooling or limiting microcoil activation times for steering of a medical device is described. This is accomplished using a heat-mitigated design and actuation strategy using the previously mentioned 44° C. as a heating threshold for safe navigation within the body (assuming no arterial flow; worst-case scenario).

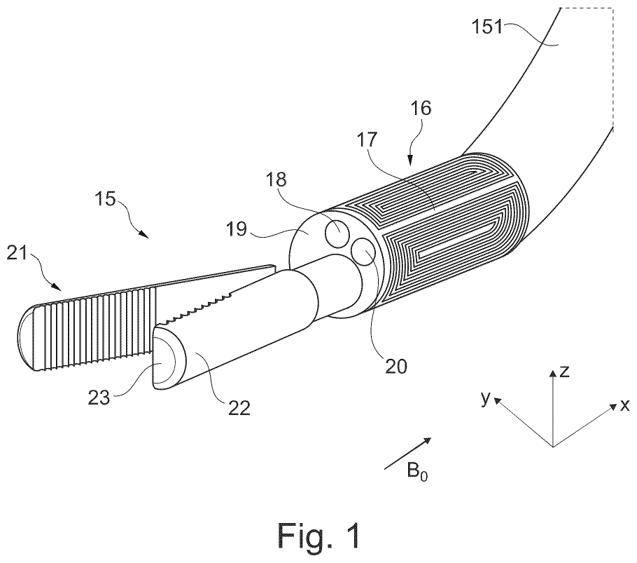

In the following an endoscope 15, here a neuroendoscope, that makes use of a quad coil design is described in detail with respect to FIGS. 1 to 5. A cartesian coordinate system is shown in FIG. 1 comprising a X-axis, a Y-axis and a Z-Axis, wherein an angle between these axes is 90°, respectively. A magnetic field comprising a magnetic field vector B₀ aligned in parallel to the X-axis is present.

The endoscope 15 comprise a tip 16, a rod-shaped portion 151, a set of coils 17 surrounding the tip 16 and (in FIG. 1 not shown) power wires 13 arranged to supply the set of coils 17 with electrical energy. The tip 16 of the endoscope 15 comprises a camera 18, an illumination device 19, optionally comprising an LED, and an opening 20 of an irrigation channel extending through the endoscope 15.

The endoscope 15 comprises an end effector 21, here a grasper, connected to the tip 16 of the endoscope 15. The end effector 21 is Lorentz force-actuated. Therefore, the grasper 21 comprises a further set of coils 22 for actuating jaws 23 of the grasper 21.

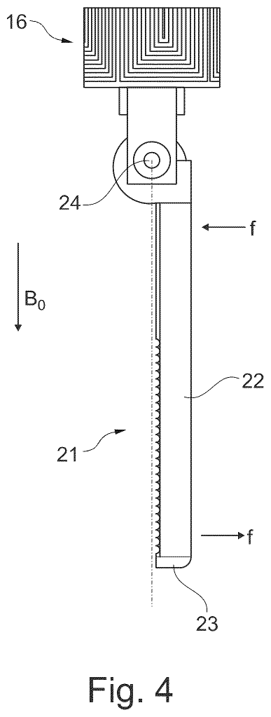

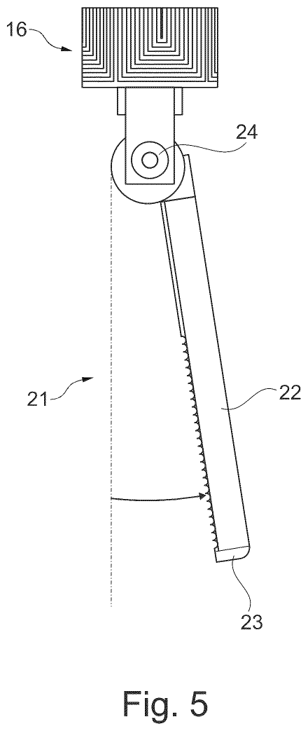

This is shown in detail in FIGS. 4 and 5. The two jaws 23 (only one is shown in FIGS. 4 and 5) are connected pivotably via a pivot joint 24 to each other. The further set of coils 22 comprises a side coil arranged at each one of the two jaws 23 such that by applying a current to the side coils of the further set of coils 22 the jaws 23 may be opened and/or closed by turning them around the pivot joint 24 (FIG. 4 shows the closed state and FIG. 5 the open state).

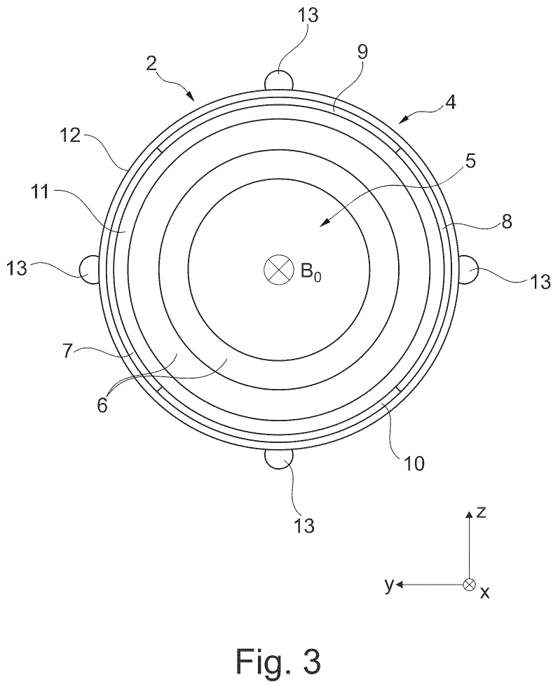

As can be gathered from FIG. 3, which is a cross sectional view of the cylindrical tip 16, the endoscope 15 comprises a cylindrical inner part 5, which may be used a working channel, having a round cross section and extending from the tip 16 through the rod-shaped portion 151 such that power wires 13, the camera 18, the illumination device 19 and the irrigation channel may be housed in the endoscope 15.

As in FIG. 1, also in FIG. 3 a cartesian coordinate system is shown comprising a X-axis, a Y-axis and a Z-Axis, wherein an angle between these axes is 90°, respectively. The X-axis is arranged in parallel to a longitudinal direction of the tip 16.

The set of coils 17 comprises two axial coils 6 having a round cross section and which are arranged around the inner part 5, wherein a center of these two axial coils 6 is located on the X-axis, i.e., windings of the axial coils 6 are wound around the X-axis. In other words, a straight line extending in parallel to the longitudinal direction of the tip 16 crosses the center of the two axial coils 6. Therefore, in the initial position of the endoscope 16 shown in FIGS. 1 and 3, where the magnetic field vector B₀ extends in parallel to the X-axis, no Lorenz force may be generated by the axial coils 6.

The set of coils 17 comprises four side coils 7-10 arranged around the two axial coils 6 of tip 16 such that a straight line extending orthogonally to the longitudinal direction of the tip 16, i.e., a straight line arranged in the YZ-plane and extending orthogonally to the X-axis, crosses a center of the respective side coil 7-10. Therefore, in the initial position of the endoscope 15 shown in FIGS. 1 and 3, where the magnetic field vector B₀ extends in parallel to the X-axis, a Lorenz force may be generated by the side coils 7-10 when applying a current to them. To do so, the endoscope 15 comprises power wires 13 arranged at an outer circumference of a isolation material 12 (which surrounds the side coils 7-10) of the tip 16 to supply the set of coils 17 with electrical energy.

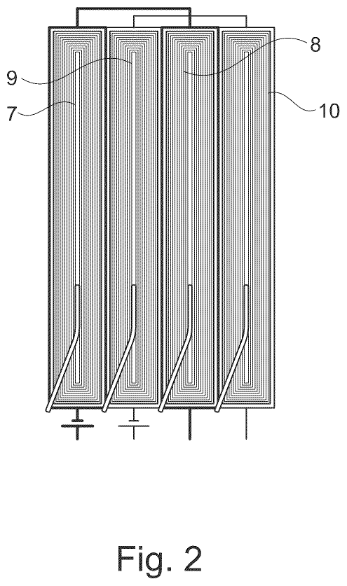

All side coils 7-10 are arranged on the same flexible circuit board 14 which is wrapped around the axial coils 6, wherein a polymide layer 11 is arranged between the side coils 7-10 and the axial coils 6 (see FIG. 3). As can be gathered from FIG. 2, the first and the second side coils 7, 8 are connected in series and the third and the fourth the side coils 9, 10 are also connected in series. As can be gathered from FIG. 3, the first and the second side coils 7, 8 are arranged respectively on opposite sides of the tip 16 and also the third and the fourth side coils 9, 10 are arranged respectively on opposite sides of the tip 16, i.e., the first and the second side coil 7, 8 are arranged in-between the third and the fourth side coil 9, 10.

The Lorentz force that is generated by the side coils 7-10 leads to a movement of the tip 16 of the endoscope substantially in the YZ-plane. Depending on the direction/polarity of the current supplied to the side coils 7-10, the tip 16 moves to the left or the right when current is supplied to the first and the second side coil 7, 8 and up or down when current is supplied to the third and the fourth side coil 9, 10. Of course these movements may be combined, e.g., moving the tip 16 simultaneously up and to the right. Therefore, with this quad coil design, four degress of freedom may be realized.

When an angle of 90° is reached, i.e., when the rod-shaped portion 151 is bound to such an extent that the magnetic field vector B₀ and the center of side coils 7-10 are in parallel, no Lorentz force is generated by the side coils 7-10 and the axial coils 6 may be used to overcome this point.

A turn number of the four side coils 7-10 may be between 2 and 40 (i.e, including 2 and 40) or between 4 and 30 (i.e, including 4 and 30), respectively. However, in the present case the turn number for the four side coils 7-10 is 7 (seven), respectively. Together with the rectangular Archimedean spiral, in plane coil design of the side coils 7-10, which are manufactured using laser machining, a very small tip diameter may be reached.

This will be explained in detail below with respect to one specific implementation of the disclosure which is described solely for explanatory purposes and is not intended to limit the scope of the disclosure, especially the claims, in any way.

Lorentz-force actuators have proven to be effective for various robotic/medical applications due to their precision, high force output, and scalability for soft device integration. These actuators utilize external magnetic fields to generate a force directly controlled for robotic actuation. Therefore, Lorentz-force actuators can utilize the high external magnetic field such as generated in MRI environments to develop robotic devices.

In this approach, controlling microcoil current polarity directly translates to a tip deflection of the tip 16 in the respective direction. The generated magnetic moment, m, and corresponding torque, T, can be determined in terms of the number of coil loops, N, current, I, and area normal vector, A, of a coil loop, and magnetic field vector (e.g., within an MR scanner) (B₀=(0, 0, B₀)), where B₀ is the (e.g., uniform, permanent, i.e., static) magnetic field (e.g., inside the MRI). This relation is represented as (Equation 1)

$$T = m \times {B}_{0} = NI ( A \times {B}_{0} )$$

which can be used to govern axial coil torque for a Lorentz force-actuated endoscope with equally-sized coil loops. In terms of saddle (side) coil implementation, microcoils can be integrated to the endoscope tip for additional degrees of freedom (DoF). Design optimization of an (e.g., MRI-driven) endoscope 15 using a four coil configuration allowed for maximizing the achievable workspace (e.g., within the brain) given certain constraints such as the number of coil sets and current inputs.

However, maximizing the number of coil turns and area may not be feasible for navigating within narrow vasculature. In order to improve upon both existing design schemes, laser machining may be used in conjunction with the Archimedean spiral coil design to create an in-plane, quad-configuration, microcoil design shown in FIG. 2.

The proposed design enables more compact and significantly smaller final endoscope diameters than previously proposed designs while achieving comparable steerability. In this approach, both saddle/side coil sets are integrated on the same circumferential plane without introducing additional layer thickness compared to the state of the art.

The governing equations for a rectangular Archimedean spiral are given below for estimating the microcoil's magnetic moment. The approximate effective area of all coil loops 7-10 can be expressed as (Equation 2)

$${A}_{total} = {\sum }_{i = 0}^{N - 1} ( {L}_{c} - w - i ( w + 2 t ) ) ( {W}_{c} - w - i ( w + 2 t ) )$$

and corresponding total wire length for estimating power consumption as (Equation 3)

$${L}_{coil} = {\sum }_{i = 0}^{N - 1} 2 ( {W}_{c} + {L}_{c} - 2 i ( 2 t + w ) - 2 w )$$

Inserting Equation (2) into Equation (1) yields the magnetic moment of a single saddle coil

m=IAₜₒₜₐₗ

Achieving higher bending angles for Lorentz force-based actuation implies maximizing the coil's magnetic moment. As shown in the above equations, tuning various parameters (i.e., coil turn number, current, endoscope diameter) can influence the performance. Typically, a larger coil turn number implies better bending performance due to the increasing coil area. However, when power is constrained to the heating threshold (e.g., 0.5 W) to mitigate heating effects, an inverse relation exists between the magnetic moment and coil turn number. In other words, a lower coil turn number is ideal for mitigating heat but requires higher currents to generate such magnitudes of electromagnetic torque leading to undesirable heating within the power wires 13. However, using larger power wires 13 to mitigate heating increases flexural rigidity depending upon wire gauge and thus has undesirable effects on endoscope steerability. Therefore, a microcoil turn number in the above description ranges, especially of approximately 7, may be used to maximize the magnetic moment while remaining within well-established current ratings for power wires 13 and an acceptable range of stiffness for the endoscope 15.

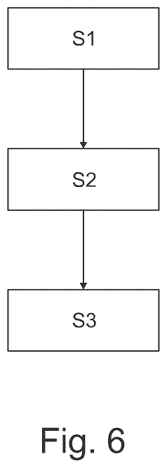

In the following a method for controlling a movement of the above described endoscope 15 in a magnetic field is described in detail. A flow chart of the method is shown in FIG. 6. The method is beneficial since Joule heating may remain a concern even with the above described design of the endoscope 15 and therefore optimizing microcoil (saddle/axial) power distribution may impact the performance of the endoscope 15.

The movement of the tip 16 comprises a deformation of said rod-shaped portion 151 resulting in a movement of the tip 16 of said rod-shaped portion 151.

In a first step S1 of the method a torque that needs to be applied onto the endoscope 15 is determined such that the tip 16 of the endoscope 15 carries out the movement.

Determining the torque may comprise solving a first optimization problem to minimize the torque to be applied onto the endoscope 15 such that the tip 16 of the endoscope 15 carries out the movement.

Solving the first optimization problem may comprise determining the deformation of said rod-shaped portion 151 that is needed for the movement of the tip 16 of said rod-shaped portion 151 from an actual location to a desired location such that a torque that is required for the deformation of said rod-shaped portion 151 is minimized. The torque that needs to be applied onto the endoscope 15 such that the endoscope 15 carries out the movement is then set to be equal to the torque that is required for the deformation of said rod-shaped portion 151.

The deformation that is needed for the movement of the tip 16 of said rod-shaped portion 151 from the actual location to the desired location may be determined using a model, optionally a Cosserat model, depicting the nonlinear dynamics of said rod-shaped portion 151.

This will be explained in detail below with respect to one specific implementation of the disclosure which is described solely for explanatory purposes and is not intended to limit the scope of the disclosure, especially the claims, in any way.

Steerable endoscopes undergo large deformations/motions during surgical procedures. One method of modeling such motions commonly used to model elastic rods and continuum rods is the Cosserat rod theory. The Cosserat rod model integrates the traditional bending and twisting of Kirchhoff rods with additional stretching and shearing to capture full beam dynamics. The Cosserat model accurately depicts the nonlinear dynamics of elastic rods with different materials and geometries. The endoscope 15 may be modeled as a cantilever beam undergoing an external torque and tip force. The state of the endoscope may be described using a set of N discretized segments Y=[y₀^{T}, y₁^{T}, . . . , yₙ^{T}]_{N}^{T}. The discretized state vector for each segment i contains segment position (pᵢ∈ [symbol] ³), orientation (Rᵢ∈SO(3)), extension force (n∈ [symbol] ³), and shear torque (m∈ [symbol] ³), which can be expressed in one vector yᵢ=[pᵢ, Rᵢ, nᵢ, mᵢ]. The rotation matrix is defined in the (e.g., MRI's) fixed coordinate frame, along with two additional coordinate frames L; control frame C representing the endoscope free length starting position and tip frame T locating the start of the microcoils. Therefore, a system of nonlinear ordinary differential equations (ODEs) can be expressed as (Equations 5 to 8)

$${\dot{p}}_{i} = {R}_{i} {v}_{i}$$

$${\dot{R}}_{i} = {R}_{i} {u}_{i}$$

$${\dot{n}}_{i} = - ρ Ag$$

$${\dot{m}}_{i} = - {\dot{p}}_{i} \times {n}_{i}$$

where v and u are tangent and curvature vectors defined as, v=\hat{z}+K₁R^{T} n and u=K₂R^{T} m m, where [symbol] ᵢ is the unit vector in local coordinate frame, K₁=diag(GA,GA,EA) and K₂=diag(EI_{A}, EI_{A},GJ). G, A, E, I_{A}, and J represent the shear modulus, cross-sectional area, elastic modulus, area moment of inertia, and polar moment of inertia, respectively. Endoscope forward kinematics, Y=f(n₀, m₀), can be calculated through numerical integration using a fourth order Runge-Kutta algorithm, given the endoscope's initial conditions: R₀=R_{C}, p₀=p_{C}, n₀=n_{C}, m₀=m_{C}. Although the forward kinematic model in an initial value problem form is useful for simulating endoscope Archimedean motion given a base wrench, an inverse kinematic model is needed to determine the minimum endoscope torque for reaching desired orientations. An inverse kinematic model in a boundary value problem (BVP) form may be formulated with the following boundary conditions: R₀=R_{C}, p₀=p_{C}, n_{τ}=0, m_{τ}=τ_{des} and R_{τ}=R_{des}. Here, n_{τ} and m_{τ} are expressed as the magnetic wrench at the tip 16 of the endoscope 15. Due to the negligible magnetic gradient pulling force acting on the tip 16 in comparison to the magnitude of a distributed Lorentz force, it is assumed there is only torque at the tip 16. Therefore, the inverse kinematic for desired tip torque (τ_{des}=IK(R_{des})) is calculated by solving the following optimization problem for tip torque (Equation 9)

$${arg min}_{Y} {‖ {R}_{𝒯 , des} {R}_{𝒯} ‖}_{2}^{2} + {‖ {τ}_{des} ‖}_{2}^{2}$$

$$s . t . Y = f ( {n}_{0} , {m}_{0} )$$

where the box minus ( [symbol] : SO(3)×SO(3)→ [symbol] ³) is the rotation difference operator based on the matrix logarithm defined in Lie algebra. Tip torque is τ_{des}=m_{N}. Optimization is solved in real-time using the iterative Levenberg-Marquardt method implemented in C++, where endoscope forward kinematics is used as the shooting function. It is important to note that the [symbol] error may be essential for the stability of the solution for near singular values, and the quadratic on tip torque regularizes the cost function to eliminate inverse kinematic solutions with loops.

In a second step S2 of the method a minimum current that needs to be supplied to each coil 6, 7-10 of the set of coils 4, 17, to reach the determined torque (τ_des) by solving a second optimization problem is determined, respectively.

This will be explained in detail below with respect to one specific implementation of the disclosure which is described solely for explanatory purposes and is not intended to limit the scope of the disclosure, especially the claims, in any way.

Microcoil-based heat generation can be reduced by optimally distributing current to the side and axial coils 6, 7-10. The tip orientation controller therefore comprises a two-stage optimization scheme: 1) inverse kinematics to determine torque using Equation (9), and 2) saddle/axial coil current distribution. A power-optimized current distribution problem is formulated as a non-linear quadratic optimization (Equations 10 and 11)

$${I}^{*} = {arg min}_{I} {‖ {τ}_{coils} - {τ}_{des} ‖}^{2} + α {‖ I ‖}_{R}^{2}$$

$$s . t . {τ}_{coils} = {τ}_{side , 1} + {τ}_{side , 2} + {τ}_{axial}$$

where I=[I_{side,1}, I_{side,2}, Iₐₓᵢₐₗ] represents the saddle/sie and axial coil currents, and τ_{coil} represents the total torque generated by a saddle and axial coil set 6, 7-10, respectively. The first term of the cost function is for consistency between desired tip torque and total coil torque, and second term is the coil power consumption cost, where R=diag(R_{side,1}, R_{side,2}, Rₐₓᵢₐₗ] is the resistance of the coils. Due to the difference in magnitude between torque error and induced currents, an α constant is incorporated (determined using a grid search to find the best fitting; 1×10-6). This optimization is also solved using the Levenberg-Marquardt method. In a comparison between actuating coils using equally-distributed power versus the optimal approach a significant amount of conserved power regardless of initial orientation of the tip 16 may be demonstrated. Such power conservation improves overall endoscope safety during steering at low rotation angles, and increases the endoscope workspace.

In a third step S3 of the method the determined minimum current (I_side1, I_side2, I_axial) is supplied to each coil of the set of coils 6, 7-10, respectively, such that the tip 16 of the endoscope 15 carries out the movement.

The third step S3 may comprise actuating the end effector 21 thereof by supplying a current to the further set of coils 22. In case the end effector 21 comprises the grasper, actuating the end effector 21 may comprise opening and/or closing the jaws 23 of the grasper 21 by applying the current to the further set of coils 22. The opening may be a movement where the jaws of the grasper are moved away from each other by turning them around the pivot joint 24 and the closing may be a movement where the jaws 23 of the grasper 21 are moved towards each other by turning them around the pivot joint 24. The method may comprise ablating and/or killing tissue, optionally comprising tumor cells, such as cancerous brain tumor cells, using the Joule heating caused by the current supplied to the further set of coils 22 for actuating the end effector 21, optionally for opening and closing the jaws 23 of the grasper 21.

The Joule heating caused by actuating the grasper 21 may be used in the third step S3 of the method for cauterization. More specifically, the cauterization comprises actuating the end effector 21 by applying a current to the further set of coils 22, and ablating and/or killing tissue, optionally comprising tumor cells, using the Joule heating caused by the current supplied to the further set of coils 22 for actuating the end effector 21.

This will be explained in detail below with respect to one specific implementation of the disclosure which is described solely for explanatory purposes and is not intended to limit the scope of the disclosure, especially the claims, in any way.

With the proposed design of the MRI-driven endoscope 15 described above, leveraging the high (3-7 T), external magnetic field of an MR scanner for heat-mitigated steering within the ventricular system of the brain becomes possible. The Lorentz force-based grasper 21 may be used for diseased tissue manipulation and ablation, i.e., cauterization. Feasibility studies show the neuroendoscope 15 can be steered precisely within the lateral ventricle to locate a tumor using both MRI and endoscopic guidance. Results also indicate grasping forces as high as 31 mN are possible and power inputs as low as 0.69 mW can cause cancerous tissue ablation.

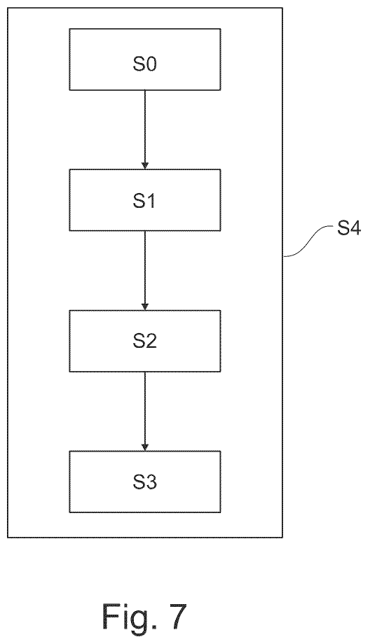

In FIG. 7 a flowchart for a flow diagram of a further method for controlling the movement of the endoscope 15 in a static magnetic field produced by a medical imaging device, here a magnetic resonance imaging device. The method comprises the above described steps S1-S3. The method further comprises in an initial step SO receiving user input with respect to the movement via a user interface, optionally comprising a joystick. The user interface may be connected to the endoscope 15. The method further comprises a fourth step S4 carried out simultaneously to the steps S0-S4, wherein this step S4 comprises determining an actual position of the endoscope 15, optionally the tip 16 thereof, using medical imaging, here the MRI, continuously and displaying the determined position of the endoscope 15 continuously on a display device with respect to a tissue, optionally of a human being or an animal, in which the endoscope 15 is located. The display device may be connected to the endoscope 15.

What is claimed is:

1-36. (canceled)

37. An endoscope, optionally a neuroendoscope, wherein the endoscope comprises:
a tip, optionally comprising a working channel extending through the tip of the endoscope,
a set of coils surrounding the tip, and
power wires arranged to supply the set of coils with electrical energy,
wherein the set of coils comprises four side coils arranged around the tip such that a straight line extending orthogonally to a longitudinal direction of the tip crosses a center of a respective side coil of the four side coils.

38. The endoscope according to claim 37, wherein:
a first side coil and a second side coil are connected in series,
a third side coil and a fourth side coil are connected in series,
the first side coil and the second side coil are arranged respectively on opposite sides of the tip, and
the third side coil and the fourth side coil are arranged respectively on opposite sides of the tip and in-between the first side coil and the second side coil.

39. The endoscope according to claim 37, wherein a turn number of at least one of the four side coils is between 2 and 40, optionally 4 and 30, further optionally 7.

40. The endoscope according to claim 37, wherein the set of coils includes at least one axial coil arranged around the tip such that a straight line extending in parallel to the longitudinal direction of the tip crosses a center of the at least one axial coil.

41. The endoscope according to claim 37, wherein at least one of the coils of the set of coils:
was manufactured using laser machining, laser lithography and/or was manually wound, and/or
has, an optionally rectangular, Archimedean spiral coil design, and/or has an in-plane design.

42. The endoscope according to claim 37, wherein the four side coils are arranged on a same, optionally flexible, circuit board.

43. The endoscope according to claim 37, wherein the endoscope includes an end effector connected to the tip of the endoscope, optionally wherein the end effector comprises a further set of coils for actuating the end effector.

44. The endoscope according to claim 43, wherein the end effector of the endoscope is configured to be actuated by applying a current to the further set of coils.

45. The endoscope according to claim 44, wherein:
the end effector includes a grasper with two jaws connected pivotably to each other, and
the further set of coils comprises at least one side coil arranged at each one of the two jaws, respectively,
optionally wherein the jaws of the grasper of the end effector are configured to be opened and/or closed by applying the current to the further set of coils.

46. The endoscope according to claim 37, wherein the tip of the endoscope comprises:
a camera,
an illumination device, optionally comprising an LED, and/or
an opening of an irrigation channel extending through the endoscope.

47. A method for controlling a movement of the endoscope according to claim 37 in a magnetic field, wherein the method comprises:
determining a torque that needs to be applied onto the endoscope such that the endoscope carries out the movement, wherein determining the torque optionally includes solving a further optimization problem to minimize the torque to be applied onto the endoscope such that the endoscope carries out the movement;
determining a current, optionally a minimum current, that needs to be supplied to the set of coils, optionally to each coil of the set of coils, respectively, to reach the determined torque by solving an optimization problem; and
supplying the determined current to the set of coils such that the endoscope carries out the movement.

48. The method according to claim 47, wherein the optimization problem is defined as follows:
$${I}^{*} = {arg min}_{I} {‖ {τ}_{coils} - {τ}_{des} ‖}^{2} + α {‖ I ‖}_{R}^{2}$$
wherein:
I represents a current supplied to each coil of the set of coils, respectively,
τ_{coils} represents a total torque generated by the set of coils when being supplied with the current I,
τ_{des} represents the torque that needs to be applied onto the endoscope such that the endoscope carries out the movement, and
R represents a resistance of each coil of the set of coils.

49. The method according to claim 47, wherein the endoscope includes a flexible substantially rod-shaped portion and the movement includes a deformation of said rod-shaped portion resulting in a movement of the tip of said rod-shaped portion, wherein solving the further optimization problem optionally includes:
determining the deformation of said rod-shaped portion that is needed for the movement of the tip of said rod-shaped portion from an actual location to a desired location such that a torque that is required for the deformation of said rod-shaped portion is minimized, and
determining the torque that needs to be applied onto the endoscope such that the endoscope carries out the movement to be equal to the torque that is required for the deformation of said rod-shaped portion.

50. The method according to claim 49, wherein the deformation that is needed for the movement of the tip of said rod-shaped portion from the actual location to the desired location is determined using a model, optionally a Cosserat model, depicting a nonlinear dynamics of said rod-shaped portion.

51. The method according to claim 47, wherein the method comprises:
receiving user input with respect to the movement via a user interface of the endoscope, the user interface optionally including a joystick,
determining an actual position of the endoscope, optionally the tip thereof, using medical imaging, and automated controlling of the movement based on the determined actual position, and/or
displaying an actual position of the endoscope, optionally the tip thereof, and/or a position of the endoscope, optionally the tip thereof, after carrying out the movement on a display device, optionally with respect to a tissue, optionally of a human being or an animal.

52. The method according to claim 47, wherein the magnetic field is produced by a medical imaging device, optionally a magnetic resonance imaging device, and/or the magnetic field is a static magnetic field.
